(19) United States
(12) Patent Application Publication
(10) Pub. No.: US 2026/0325515 A1
(43) Pub. Date: Oct. 22, 2026

(54) TREATMENT OF CANINE CANCERS

(71) Applicant: OneHealthCompany, Inc., Palo Alto, CA (US)

(72) Inventors: Christina Kelly Lopes, Palo Alto, CA (US); Gerald Post, Palo Alto, CA (US); Lucas Rodrigues, Palo Alto, CA (US); Garrett Harvey, Palo Alto, CA (US)

(21) Appl. No.: 19/678,247

(22) Filed: May 15, 2026

Related U.S. Application Data
(63) Continuation of application No. PCT/US2024/056257, filed on Nov. 15, 2024.
(60) Provisional application No. 63/599,677, filed on Nov. 16, 2023.

(51) Int. Cl. A61K 31/502; A61K 31/167; A61K 31/282; A61K 31/404; A61K 31/436; A61K 31/44; A61K 31/4545; A61K 31/506; A61K 31/517; A61K 31/519; A61K 31/5377; A61P 35/00; C12Q 1/6886

(52) U.S. Cl. CPC A61K 31/502; A61K 31/167; A61K 31/282; A61K 31/404; A61K 31/436; A61K 31/44; A61K 31/4545; A61K 31/506; A61K 31/517; A61K 31/519; A61K 31/5377; A61P 35/00; C12Q 1/6886; C12Q 2600/156

(57) ABSTRACT

Described herein are methods useful for the treatment of cancers in a canine subject with a pharmaceutical compositions with an anti-cancer agent (e.g., Olaparib, Toceranib, and Palbociclib).

### CROSS-REFERENCE

This application is a continuation of International (PCT) patent application No. PCT/US2024/056257, filed on Nov. 15, 2024, which claims the benefit of and priority to U.S. Provisional Patent Application No. 63/599,677, filed Nov. 16, 2023, the disclosure of each of which are hereby incorporated by reference in their entirety for all purposes.

### BACKGROUND

Disclosed herein are methods and compositions useful for the treatment of cancers in subjects with various compositions comprising targeted anti-cancer agents, including Olaparib, Toceranib, and Palbociclib.

Although targeted anti-cancer agents have been effective in the treatment of some human cancers, these agents are generally exhibit unpredictable efficacy, especially. in canines. Therefore, there is a need for improved methods and compositions for predicting efficacy and treating cancers in subjects with targeted anti-cancer agents.

### SUMMARY

Described herein, in certain embodiments, are methods of treating osteosarcoma in a canine subject, comprising: administering to the canine subject in need thereof a therapeutically effective amount of a pharmaceutical composition comprising Olaparib. In some embodiments, the Olaparib is administered at a dose equal to or less than 5 mg/kg. In some embodiments, the Olaparib is administered at a dose of 3 mg/kg. In some embodiments, the Olaparib is administered at a frequency selected from the group consisting of, twice daily, once daily, once every other day, once every third day, once every fourth day, once every 5th day, or weekly. In some embodiments, the Olaparib is administered orally. In some embodiments, the method further comprises administering a therapeutically effective amount of at least one additional anti-cancer agent. In some embodiments, the at least one additional anti-cancer agent is a DNA damaging chemotherapeutic agent. In some embodiments, the DNA damaging chemotherapeutic agent is selected from the group consisting of: a DNA-alkylating agent, DNA crosslinking agent, antimetabolite, topoisomerase inhibitor and a DNA intercalating agent. In some embodiments, the at least one additional anti-cancer agent is a targeted anti-cancer agent. In some embodiments, the targeted anti-cancer agent is selected from the group consisting of: Crizotinib, Erlotinib, Gefinitib, Imatinib, Dasatinib, Rapamycin, Sorafenib, Trametinib, and Vorinostat. In some embodiments, the at least one additional anti-cancer agent is carboplatin. In some embodiments, the method further comprises performing surgery on the subject. In some embodiments, the method further comprises administering to the subject ionizing radiation. In some embodiments, the osteosarcoma harbors at least one mutation in at least one gene selected from the group consisting of p53 and BRCA1. In some embodiments, the method further comprises having determined from a biological sample derived from the osteosarcoma, that the osteosarcoma harbors a mutation in at least one gene selected from the group consisting of p53 and BRCA1. In some embodiments, the biological sample is a nucleic acid sample. In some embodiments, the biological sample is a purified nucleic acid sample. In some embodiments, the sample is DNA. In some embodiments, the sample is RNA. In some embodiments, the determining of the mutation in the at least one gene is performed by sequencing the nucleic acid sample.

Described herein, in certain embodiments, are methods of treating soft tissue sarcoma in a canine subject, comprising: administering to the canine subject in need thereof a therapeutically effective amount of a pharmaceutical composition comprising Olaparib. In some embodiments, the Olaparib is administered at a dose equal to or less than 5 mg/kg. In some embodiments, the Olaparib is administered at a dose of 3 mg/kg. In some embodiments, the Olaparib is administered at a frequency selected from the group consisting of, twice daily, once daily, once every other day, once every third day, once every fourth day, once every 5th day, or weekly. In some embodiments, the Olaparib is administered orally. In some embodiments, the method further comprises administering a therapeutically effective amount of at least one additional anti-cancer agent. In some embodiments, the at least one additional anti-cancer agent is a DNA damaging chemotherapeutic agent. In some embodiments, the DNA damaging chemotherapeutic agent is selected from the group consisting of: a DNA-alkylating agent, DNA crosslinking agent, antimetabolite, topoisomerase inhibitor and a DNA intercalating agent. In some embodiments, the at least one additional anti-cancer agent is a targeted anti-cancer agent. In some embodiments, the targeted anti-cancer agent is selected from the group consisting of: Crizotinib, Erlotinib, Gefinitib, Imatinib, Dasatinib, Rapamycin, Sorafenib, Trametinib, and Vorinostat. In some embodiments, the at least one additional anti-cancer agent is carboplatin. In some embodiments, the method further comprises performing surgery on the subject. In some embodiments, the method further comprises administering to the subject ionizing radiation. In some embodiments, the osteosarcoma harbors at least one mutation in at least one gene selected from the group consisting of p53 and BRCA1. In some embodiments, the method further comprises having determined from a biological sample derived from the osteosarcoma, that the osteosarcoma harbors a mutation in at least one gene selected from the group consisting of p53 and BRCA1. In some embodiments, the biological sample is a nucleic acid sample. In some embodiments, the biological sample is a purified nucleic acid sample. In some embodiments, the sample is DNA. In some embodiments, the sample is RNA. In some embodiments, the determining of the mutation in the at least one gene is performed by sequencing the nucleic acid sample.

Described herein, in certain embodiments, are methods of treating squamous cell carcinoma in a canine subject, comprising: administering to the canine subject in need thereof a therapeutically effective amount of a pharmaceutical composition comprising Toceranib. In some embodiments, the Toceranib is administered at a dose equal to or less than 5 mg/kg. In some embodiments, the Toceranib is administered at a dose 2 mg/kg to 3 mg/kg. In some embodiments, the Toceranib is administered at a frequency selected from the group consisting of, twice daily, once daily, once every other day, once every third day, once every fourth day, once every 5th day, or weekly. In some embodiments, the Toceranib is administered once three times a week. In some embodiments, the Toceranib is administered orally. In some embodiments, the method further comprises administering a therapeutically effective amount of at least one additional anti-cancer agent. In some embodiments, the at least one additional anti-cancer agent is a DNA damaging chemotherapeutic agent. In some embodiments, the DNA damaging chemotherapeutic agent is selected from the group consisting of: a DNA-alkylating agent, DNA crosslinking agent, antimetabolite, topoisomerase inhibitor and a DNA intercalating agent. In some embodiments, the at least one additional anti-cancer agent is a targeted anti-cancer agent. In some embodiments, the targeted anti-cancer agent is selected from the group consisting of: Crizotinib, Erlotinib, Gefinitib, Imatinib, Dasatinib, Rapamycin, Sorafenib, Trametinib, and Vorinostat. In some embodiments, the method further comprises performing surgery on the subject. In some embodiments, the method further comprises administering to the subject ionizing radiation.

Described herein, in certain embodiments, are methods of treating melanoma in a canine subject, comprising: administering to the canine subject in need thereof a therapeutically effective amount of a pharmaceutical composition comprising Palbociclib. In some embodiments, the Palbociclib is administered at a dose equal to or less than 0.2 mg/kg. In some embodiments, the Palbociclib is administered at a dose of 0.05 mg/kg to 0.4 mg/kg. In some embodiments, the Palbociclib is administered at a frequency selected from the group consisting of, twice daily, once daily, once every other day, once every third day, once every fourth day, once every 5th day, or weekly. In some embodiments, the Palbociclib is administered orally. In some embodiments, the method further comprises administering a therapeutically effective amount of at least one additional anti-cancer agent. In some embodiments, the at least one additional anti-cancer agent is a DNA damaging chemotherapeutic agent. In some embodiments, the DNA damaging chemotherapeutic agent is selected from the group consisting of: a DNA-alkylating agent, DNA crosslinking agent, antimetabolite, topoisomerase inhibitor and a DNA intercalating agent. In some embodiments, the at least one additional anti-cancer agent is a targeted anti-cancer agent. In some embodiments, the targeted anti-cancer agent is selected from the group consisting of: Crizotinib, Erlotinib, Gefinitib, Imatinib, Dasatinib, Rapamycin, Sorafenib, Trametinib, and Vorinostat. In some embodiments, the method further comprises performing surgery on the subject. In some embodiments, the method further comprises administering to the subject ionizing radiation.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “FIGURE” and “FIG.” herein), of which:

FIG. 1 depicts a graph demonstrating dogs with oral malignant melanoma carrying BRCA1/2 mutations treated with Olaparib (gray, 1001), with median survival time of 448 days, compared to dogs with different genomic alterations and treatment with the median survival time of 235 days (P=0.0120) (black, 1003).

### DETAILED DESCRIPTION

Briefly, and as described in more detail below, described herein are methods useful for the treatment of cancer in a subject (e.g., canine subject) with a pharmaceutical compositions comprising anti-cancer agents, including Olaparib, Toceranib, and Palbociclib, and combinations thereof. Also described herein are methods for identification or selection of subjects with cancers that are likely to derive significant therapeutic responses to administration of the pharmaceutical compositions comprising anti-cancer agents, including Olaparib, Toceranib, and Palbociclib. The methods of the present disclosure are useful for treating subjects with cancer with targeted therapies comprising Olaparib, Toceranib, and Palbociclib, and combinations thereof for increasing survival of the subjects compared to traditional therapies. In certain aspects, the methods herein cause increased survival of a subject with cancer following treatment with the targeted therapies disclosed herein compared to traditional chemotherapy.

### DEFINITIONS

Terms used in the claims and specification are defined as set forth below unless otherwise specified.

The term “canine cancer” can refer to any cancer, tumor or hyperproliferative disorder that is present in a canine subject. In embodiments, the canine cancer is a specified cancer that is surprisingly responsive to one or more of the treatments described in this disclosure.

The term “treating” refers to any therapeutically beneficial result in the treatment of a disease state, e.g., a cancer disease state, including lessening in the severity or progression, remission, or cure thereof.

The term “sufficient amount” means an amount sufficient to produce a desired effect, e.g., an amount sufficient to modulate protein aggregation in a cell.

The term “therapeutically effective amount” is an amount that is effective to ameliorate a symptom of cancer or hyperproliferative disease.

The term “anti-cancer agent” refers to, but is not limited to chemotherapeutic agents, targeted therapies, hormones, and immunotherapies. Anti-cancer agents can be, but are not limited to, a small molecule, an antibody or antibody fragment, nucleic acid (e.g., DNA or RNA), carbohydrates, peptides, lipids, exosomes, cells, or combinations thereof.

The terms “recipient”, “individual”, “subject”, “host”, and “patient”, are used interchangeably herein and in some embodiments, refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. In some embodiments, the mammal is human. In some embodiments, the mammal is a canine. None of these terms require the supervision of medical personnel.

The term “targeted anti-cancer agent” refers to, but is not limited to, anti-cancer agents that specifically alter a cancer cell characteristic (e.g., an agent that interacts with a gene product, such as a protein or RNA, that regulates a cancer cell characteristic, such as cell proliferation, cell survival, metastasis, immune evasion, etc.).

The term “overexpression” when referring to a gene (e.g., an oncogene such as BRAF), refers to increased amounts of mRNA or protein with respect to a non-cancer tissue control sample. A gene can be considered overexpressed when the mRNA and/or protein amounts of the gene are greater than 2.0 fold, 5 fold, 10 fold, 20 fold, 30 fold, 40 fold, 50 fold, 60 fold, 70 fold, 80 fold, 90 fold, 100 fold, 150 fold, 200 fold 300 fold, 400 fold 500 fold or 1000 fold the amount of RNA and/or protein of a non-cancer tissue control sample. In certain embodiments, the gene is overexpressed when the mRNA and/or protein amounts of the gene are 2 fold to 10 fold, 5 fold to 10 fold, 10 fold to 100 fold, 10 fold to 50 fold, 50 fold to 100 fold, or 100 fold to 1000 fold greater than the amount of RNA and or protein of a non-cancer tissue control sample. Overexpression can be determined by any appropriate method known in the art, including, but not limited to, RNA SEQ, and quantitative PCR, immunohistochemistry.

The term “underexpression” when referring to a gene (e.g., a tumor suppressor gene such as p53), refers to decreased amounts of mRNA or protein with respect to a non-cancer tissue control sample. A gene can be considered under expressed when the mRNA and/or protein amounts of the gene are less than 2.0 fold, 5 fold, 10 fold, 20 fold, 30 fold, 40 fold, 50 fold, 60 fold, 70 fold, 80 fold, 90 fold, 100 fold, 150 fold, 200 fold 300 fold, 400 fold 500 fold or 1000 fold the amount of RNA and/or protein of a non-cancer tissue control sample. In certain embodiments, the gene is under expressed when the mRNA and/or protein amounts of the gene are 2 fold to 10 fold, 5 fold to 10 fold, 10 fold to 100 fold, 10 fold to 50 fold, 50 fold to 100 fold, or 100 fold to 1000 fold less than the amount of RNA and or protein of a non-cancer tissue control sample. Underexpression can be determined by any appropriate method known in the art, including, but not limited to, RNA SEQ, and quantitative PCR, immunohistochemistry.

It must be noted that, as used in the specification and the appended claims, the singular forms “a,” “an” and “the” include plural referents unless the context clearly dictates otherwise.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”, or “a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

### Methods

Described herein are methods of treating cancers comprising administration of a therapeutically effective amount of a pharmaceutical composition comprising Olaparib, Toceranib, and Palbociclib, and combinations thereof. In some embodiments, the cancer is a canine cancer.

### Types of Cancers

Canine cancers include, but are not limited to, solid tumors, leukemia, lymphocytic leukemia, lymphoma, sarcoma, soft tissue sarcoma, multiple myeloma, hemangiosarcoma, histiocytic sarcoma, hepatocellular carcinoma, lymphosarcoma, osteosarcoma, transitional cell carcinoma, squamous cell carcinoma, subungual squamous cell carcinoma, mammary carcinoma, melanoma, mast cell tumors, lipoma, apocrine gland anal sac adenocarcinomas (AGASACA), lung cancer, pulmonary adenocarcinoma, pancreatic cancer, stomach cancer, prostate cancer, nasal cancer, liver cancer, brain cancer, bladder cancer and thyroid cancer. The methods and compositions described herein, also can be used on any other cancers, including cancers that are rare in canines.

In some embodiments, the cancer is osteosarcoma. In some embodiments, the cancer is soft tissue sarcoma. In some embodiments, the cancer is squamous cell carcinoma. In some embodiments, the cancer is melanoma. In some embodiments, the cancer is anal sac carcinoma.

### Modes of Administration

In certain embodiments, the pharmaceutical compositions comprising the compounds described herein are administered to a subject in need thereof by oral administration. The pharmaceutical compositions can be formulated for administration to subjects by a variety of routes, including orally, intranasally, by inhalation, intramuscularly, intraperitoneally, and parenterally, including intravenously or subcutaneously. Pharmaceutical compositions can be formulated in volumes and concentrations suitable for bolus administration, for continuous infusion, or for subcutaneous administration. Pharmaceutical compositions comprising anti-cancer agents may be administered by any preferred route of administration.

### Dosing Regimens

The compounds described herein can be administered in at any suitable dose. An individual dose of the compound can be any ranging from 0.01-200 mg/kg, inclusive. An individual dose of the compound can a be 1.0-100 mg/kg, 100-200 mg/kg, 1.0-10 mg/kg, 10-20 mg/kg, 20-30 mg/kg, 30-40 mg/kg, 40-50 mg/kg; 50-60 mg/kg, 70-80 mg/kg, 80-90 mg/kg, 90-100 mg/kg, 10-100 mg/kg, 100-150 mg/kg, 150-200 mg/kg, 20-50 mg/kg, 50-100 mg/kg, 1.0-20 mg/kg, 1.0-10 mg/kg, 1.0-5.0 mg/kg, 1.0-2.0 mg/kg, 5-10 mg/kg, 10-15 mg/kg, 15-20 mg/kg, 0.5-1.5 mg/kg, 0.5-1.0 mg/kg, 0.1-1.0 mg/kg, 0.1-0.2 mg/kg, 0.2-0.3 mg/kg, 0.3-0.4 mg/kg, 0.4-0.5 mg/kg, 0.5-0.6 mg/kg, 0.6-0.7 mg/kg, 0.7-0.8 mg/kg, 0.8-0.9 mg/kg, 0.9-1.0 mg/kg, 0.01-0.1 mg/kg, 0.01-0.02 mg/kg, 0.02-0.03 mg/kg, 0.03-0.04 mg/kg, 0.04-0.05 mg/kg, 0.05-0.06 mg/kg, 0.06-0.07 mg/kg, 0.07-0.08 mg/kg, 0.08-0.09 mg/kg, 0.09-0.1 mg/kg, 1.0-3.0 mg/kg, 1.1-1.2 mg/kg, 1.2-1.3 mg/kg, 1.3-1.4 mg/kg, 1.4-1.5 mg/kg, 1.5-1.6 mg/kg, 1.6-1.7 mg/kg, 1.7-1.8 mg/kg, 1.8-1.9 mg/kg, 1.9-2.0 mg/kg, 2.0-2.1 mg/kg, 2.1-2.2 mg/kg, 2.2-2.3 mg/kg, 2.3-2.4 mg/kg, 2.4-2.5 mg/kg, 2.5-2.6 mg/kg, 2.6-2.7 mg/kg, 2.7-2.8 mg/kg, 2.8-2.9 mg/kg, or 2.9-3.0 mg/kg, inclusive.

In a variety of embodiments, the pharmaceutical compositions described herein are administered for a period of 1 day to indefinitely, a period of 1 week to 6 months, a period of 3 months to 5 years, a period of 6 months to 1 or 2 years, or the like. Optionally, administration is repeated; for example, in certain embodiments, the pharmaceutical compositions described herein are administered once daily, twice daily, three times daily, four times daily, five times daily, every two days, every three days, every five days, once a week, once every two weeks, once a month, every other month, semi-annually, or annually. In certain embodiments, the pharmaceutical compositions described herein are administered at regular intervals over a period of several weeks, followed by a period of rest, during which no pharmaceutical composition described herein is administered. For example, in certain embodiments, pharmaceutical compositions comprising a compound described herein are administered for one, two, three, or more weeks, followed by one, two, three, or more weeks without pharmaceutical composition administration. The repeated administration can be at a specified frequency; for example, in certain embodiments, the pharmaceutical composition comprising a compound described herein is administered once daily, twice daily, three times daily, four times daily, five times daily, every two days, every three days, every five days, once a week, once every two weeks, once a month, every other month, semi-annually, or annually. In certain embodiments, the pharmaceutical composition comprising a compound is administered at regular intervals over a period of several weeks, followed by a period of rest, during which no pharmaceutical composition comprising a compound is administered. For example, in certain embodiments, the pharmaceutical composition is administered for one, two, three, or more weeks, followed by one, two, three, or more weeks without administration of the pharmaceutical composition comprising the compound. The repeated administration can be at the same dose or a different dose.

In some embodiments, Olaparib is administered at a dose of about 0.01-0.1 mg/kg. In some embodiments, Olaparib is administered at a dose of about 1-20 mg/kg. In some embodiments, Olaparib is administered at a dose of about 5-10 mg/kg. In some embodiments, Olaparib is administered at a dose of about 1-5 mg/kg. In some embodiments, Olaparib is administered at a dose of about 0.01-0.05 mg/kg. In some embodiments, Olaparib is administered at a dose of about 0.02-0.03 mg/kg. In some embodiments, Olaparib is administered at a dose of about 0.1 mg/kg. In some embodiments, Olaparib is administered at a dose of about 0.02 mg/kg. In some embodiments, Olaparib is administered at a dose of about 0.03 mg/kg. In some embodiments, Olaparib is administered at a dose of about 1 mg/kg. In some embodiments, Olaparib is administered at a dose of about 2 mg/kg. In some embodiments, Olaparib is administered at a dose of about 3 mg/kg. In some embodiments, Olaparib is administered at a dose of about 4 mg/kg. In some embodiments, Olaparib is administered at a dose of about 5 mg/kg. In some embodiments, Olaparib is administered at a dose of about 6 mg/kg. In some embodiments, Olaparib is administered at a dose of about 7 mg/kg. In some embodiments, Olaparib is administered at a dose of about 8 mg/kg. In some embodiments, Olaparib is administered at a dose of about 9 mg/kg. In some embodiments, Olaparib is administered at a dose of about 10 mg/kg. In some embodiments, Olaparib is administered at a dose of about 15 mg/kg. In some embodiments, Olaparib is administered at a dose of about 20 mg/kg. In some embodiments, Olaparib is administered at a dose of about 25 mg/kg. In some embodiments, Olaparib is administered at a dose of about 30 mg/kg. In some embodiments, Olaparib is administered at a dose of about 40 mg/kg. In some embodiments, Olaparib is administered at a dose of about 50 mg/kg. In some embodiments, Olaparib is administered per day orally.

In some embodiments, the subject has osteosarcoma, and the subject is administered Olaparib at a dose of about 0.01-0.1 mg/kg. In some embodiments, the subject has osteosarcoma, and the subject is administered Olaparib at a dose of about 1-20 mg/kg. In some embodiments, the subject has osteosarcoma, and the subject is administered Olaparib at a dose of about 5-10 mg/kg. In some embodiments, the subject has osteosarcoma, and the subject is administered Olaparib at a dose of about 1-5 mg/kg. In some embodiments, the subject has osteosarcoma, and the subject is administered Olaparib at a dose of about 0.01-0.05 mg/kg. In some embodiments, the subject has osteosarcoma, and the subject is administered Olaparib at a dose of about 0.02-0.03 mg/kg. In some embodiments, the subject has osteosarcoma, and the subject is administered Olaparib at a dose of about 0.1 mg/kg. In some embodiments, the subject has osteosarcoma, and the subject is administered Olaparib at a dose of about 0.02 mg/kg. In some embodiments, the subject has osteosarcoma, and the subject is administered Olaparib at a dose of about 0.03 mg/kg. In some embodiments, the subject has osteosarcoma, and the subject is administered Olaparib at a dose of about 1 mg/kg. In some embodiments, the subject has osteosarcoma, and the subject is administered Olaparib at a dose of about 2 mg/kg. In some embodiments, the subject has osteosarcoma, and the subject is administered Olaparib at a dose of about 3 mg/kg. In some embodiments, the subject has osteosarcoma, and the subject is administered Olaparib at a dose of about 4 mg/kg. In some embodiments, the subject has osteosarcoma, and the subject is administered Olaparib at a dose of about 5 mg/kg. In some embodiments, the subject has osteosarcoma, and the subject is administered Olaparib at a dose of about 6 mg/kg. In some embodiments, the subject has osteosarcoma, and the subject is administered Olaparib at a dose of about 7 mg/kg. In some embodiments, the subject has osteosarcoma, and the subject is administered Olaparib at a dose of about 8 mg/kg. In some embodiments, the subject has osteosarcoma, and the subject is administered Olaparib at a dose of about 9 mg/kg. In some embodiments, the subject has osteosarcoma, and the subject is administered Olaparib at a dose of about 10 mg/kg. In some embodiments, the subject has osteosarcoma, and the subject is administered Olaparib at a dose of about 15 mg/kg. In some embodiments, the subject has osteosarcoma, and the subject is administered Olaparib at a dose of about 20 mg/kg. In some embodiments, the subject has osteosarcoma, and the subject is administered Olaparib at a dose of about 25 mg/kg. In some embodiments, the subject has osteosarcoma, and the subject is administered Olaparib at a dose of about 30 mg/kg. In some embodiments, the subject has osteosarcoma, and the subject is administered Olaparib at a dose of about 40 mg/kg. In some embodiments, the subject has osteosarcoma, and the subject is administered Olaparib at a dose of about 50 mg/kg. In some embodiments, Olaparib is administered per day orally.

In some embodiments, the subject has soft tissue sarcoma, and the subject is administered Olaparib at a dose of about 0.01-0.1 mg/kg. In some embodiments, the subject has soft tissue sarcoma, and the subject is administered Olaparib at a dose of about 1-20 mg/kg. In some embodiments, the subject has soft tissue sarcoma, and the subject is administered Olaparib at a dose of about 5-10 mg/kg. In some embodiments, the subject has soft tissue sarcoma, and the subject is administered Olaparib at a dose of about 1-5 mg/kg. In some embodiments, the subject has soft tissue sarcoma, and the subject is administered Olaparib at a dose of about 0.01-0.05 mg/kg. In some embodiments, the subject has soft tissue sarcoma, and the subject is administered Olaparib at a dose of about 0.02-0.03 mg/kg. In some embodiments, the subject has soft tissue sarcoma, and the subject is administered Olaparib at a dose of about 0.1 mg/kg. In some embodiments, the subject has soft tissue sarcoma, and the subject is administered Olaparib at a dose of about 0.02 mg/kg. In some embodiments, the subject has soft tissue sarcoma, and the subject is administered Olaparib at a dose of about 0.03 mg/kg. In some embodiments, the subject has soft tissue sarcoma, and the subject is administered Olaparib at a dose of about 1 mg/kg. In some embodiments, the subject has soft tissue sarcoma, and the subject is administered Olaparib at a dose of about 2 mg/kg. In some embodiments, the subject has soft tissue sarcoma, and the subject is administered Olaparib at a dose of about 3 mg/kg. In some embodiments, the subject has soft tissue sarcoma, and the subject is administered Olaparib at a dose of about 4 mg/kg. In some embodiments, the subject has soft tissue sarcoma, and the subject is administered Olaparib at a dose of about 5 mg/kg. In some embodiments, the subject has soft tissue sarcoma, and the subject is administered Olaparib at a dose of about 6 mg/kg. In some embodiments, the subject has soft tissue sarcoma, and the subject is administered Olaparib at a dose of about 7 mg/kg. In some embodiments, the subject has soft tissue sarcoma, and the subject is administered Olaparib at a dose of about 8 mg/kg. In some embodiments, the subject has soft tissue sarcoma, and the subject is administered Olaparib at a dose of about 9 mg/kg. In some embodiments, the subject has soft tissue sarcoma, and the subject is administered Olaparib at a dose of about 10 mg/kg. In some embodiments, the subject has soft tissue sarcoma, and the subject is administered Olaparib at a dose of about 15 mg/kg. In some embodiments, the subject has soft tissue sarcoma, and the subject is administered Olaparib at a dose of about 20 mg/kg. In some embodiments, the subject has soft tissue sarcoma, and the subject is administered Olaparib at a dose of about 25 mg/kg. In some embodiments, the subject has soft tissue sarcoma, and the subject is administered Olaparib at a dose of about 30 mg/kg. In some embodiments, the subject has soft tissue sarcoma, and the subject is administered Olaparib at a dose of about 40 mg/kg. In some embodiments, the subject has soft tissue sarcoma, and the subject is administered Olaparib at a dose of about 50 mg/kg. In some embodiments, Olaparib is administered per day orally.

In some embodiments, Olaparib is administered at a dose of about 0.01-0.1 mg/kg/day. In some embodiments, Olaparib is administered at a dose of about 1-20 mg/kg/day. In some embodiments, Olaparib is administered at a dose of about 5-10 mg/kg/day. In some embodiments, Olaparib is administered at a dose of about 1-5 mg/kg/day. In some embodiments, Olaparib is administered at a dose of about 0.01-0.05 mg/kg/day. In some embodiments, Olaparib is administered at a dose of about 0.02-0.03 mg/kg/day. In some embodiments, Olaparib is administered at a dose of about 0.1 mg/kg/day. In some embodiments, Olaparib is administered at a dose of about 0.02 mg/kg/day. In some embodiments, Olaparib is administered at a dose of about 0.03 mg/kg/day. In some embodiments, Olaparib is administered at a dose of about 1 mg/kg/day. In some embodiments, Olaparib is administered at a dose of about 2 mg/kg/day. In some embodiments, Olaparib is administered at a dose of about 3 mg/kg/day. In some embodiments, Olaparib is administered at a dose of about 4 mg/kg/day. In some embodiments, Olaparib is administered at a dose of about 5 mg/kg/day. In some embodiments, Olaparib is administered at a dose of about 6 mg/kg/day. In some embodiments, Olaparib is administered at a dose of about 7 mg/kg/day. In some embodiments, Olaparib is administered at a dose of about 8 mg/kg/day. In some embodiments, Olaparib is administered at a dose of about 9 mg/kg/day. In some embodiments, Olaparib is administered at a dose of about 10 mg/kg/day. In some embodiments, Olaparib is administered at a dose of about 15 mg/kg/day. In some embodiments, Olaparib is administered at a dose of about 20 mg/kg/day. In some embodiments, Olaparib is administered at a dose of about 25 mg/kg/day. In some embodiments, Olaparib is administered at a dose of about 30 mg/kg/day. In some embodiments, Olaparib is administered at a dose of about 40 mg/kg/day. In some embodiments, Olaparib is administered at a dose of about 50 mg/kg/day. In some embodiments, Olaparib is administered per day orally.

In some embodiments, the subject has osteosarcoma, and the subject is administered Olaparib at a dose of about 0.01-0.1 mg/kg/day. In some embodiments, the subject has osteosarcoma, and the subject is administered Olaparib at a dose of about 1-20 mg/kg/day. In some embodiments, the subject has osteosarcoma, and the subject is administered Olaparib at a dose of about 5-10 mg/kg/day. In some embodiments, the subject has osteosarcoma, and the subject is administered Olaparib at a dose of about 1-5 mg/kg/day. In some embodiments, the subject has osteosarcoma, and the subject is administered Olaparib at a dose of about 0.01-0.05 mg/kg/day. In some embodiments, the subject has osteosarcoma, and the subject is administered Olaparib at a dose of about 0.02-0.03 mg/kg/day. In some embodiments, the subject has osteosarcoma, and the subject is administered Olaparib at a dose of about 0.1 mg/kg/day. In some embodiments, the subject has osteosarcoma, and the subject is administered Olaparib at a dose of about 0.02 mg/kg/day. In some embodiments, the subject has osteosarcoma, and the subject is administered Olaparib at a dose of about 0.03 mg/kg/day. In some embodiments, the subject has osteosarcoma, and the subject is administered Olaparib at a dose of about 1 mg/kg/day. In some embodiments, the subject has osteosarcoma, and the subject is administered Olaparib at a dose of about 2 mg/kg/day. In some embodiments, the subject has osteosarcoma, and the subject is administered Olaparib at a dose of about 3 mg/kg/day. In some embodiments, the subject has osteosarcoma, and the subject is administered Olaparib at a dose of about 4 mg/kg/day. In some embodiments, the subject has osteosarcoma, and the subject is administered Olaparib at a dose of about 5 mg/kg/day. In some embodiments, the subject has osteosarcoma, and the subject is administered Olaparib at a dose of about 6 mg/kg/day. In some embodiments, the subject has osteosarcoma, and the subject is administered Olaparib at a dose of about 7 mg/kg/day. In some embodiments, the subject has osteosarcoma, and the subject is administered Olaparib at a dose of about 8 mg/kg/day. In some embodiments, the subject has osteosarcoma, and the subject is administered Olaparib at a dose of about 9 mg/kg/day. In some embodiments, the subject has osteosarcoma, and the subject is administered Olaparib at a dose of about 10 mg/kg/day. In some embodiments, the subject has osteosarcoma, and the subject is administered Olaparib at a dose of about 15 mg/kg/day. In some embodiments, the subject has osteosarcoma, and the subject is administered Olaparib at a dose of about 20 mg/kg/day. In some embodiments, the subject has osteosarcoma, and the subject is administered Olaparib at a dose of about 25 mg/kg/day. In some embodiments, the subject has osteosarcoma, and the subject is administered Olaparib at a dose of about 30 mg/kg/day. In some embodiments, the subject has osteosarcoma, and the subject is administered Olaparib at a dose of about 40 mg/kg/day. In some embodiments, the subject has osteosarcoma, and the subject is administered Olaparib at a dose of about 50 mg/kg/day. In some embodiments, Olaparib is administered per day orally.

In some embodiments, the subject has melanoma, and the subject is administered Olaparib at a dose of about 0.01-0.1 mg/kg/day. In some embodiments, the subject has melanoma (e.g., oral malignant melanoma), and the subject is administered Olaparib at a dose of about 1-20 mg/kg/day. In some embodiments, the subject has melanoma, and the subject is administered Olaparib at a dose of about 5-10 mg/kg/day. In some embodiments, the subject has melanoma, and the subject is administered Olaparib at a dose of about 1-5 mg/kg/day. In some embodiments, the subject has melanoma, and the subject is administered Olaparib at a dose of about 0.01-0.05 mg/kg/day. In some embodiments, the subject has melanoma, and the subject is administered Olaparib at a dose of about 0.02-0.03 mg/kg/day. In some embodiments, the subject has melanoma, and the subject is administered Olaparib at a dose of about 0.1 mg/kg/day. In some embodiments, the subject has melanoma, and the subject is administered Olaparib at a dose of about 0.02 mg/kg/day. In some embodiments, the subject has melanoma, and the subject is administered Olaparib at a dose of about 0.03 mg/kg/day. In some embodiments, the subject has melanoma, and the subject is administered Olaparib at a dose of about 1 mg/kg/day. In some embodiments, the subject has melanoma, and the subject is administered Olaparib at a dose of about 2 mg/kg/day. In some embodiments, the subject has melanoma, and the subject is administered Olaparib at a dose of about 3 mg/kg/day. In some embodiments, the subject has melanoma, and the subject is administered Olaparib at a dose of about 4 mg/kg/day. In some embodiments, the subject has melanoma, and the subject is administered Olaparib at a dose of about 5 mg/kg/day. In some embodiments, the subject has melanoma, and the subject is administered Olaparib at a dose of about 6 mg/kg/day. In some embodiments, the subject has melanoma, and the subject is administered Olaparib at a dose of about 7 mg/kg/day. In some embodiments, the subject has melanoma, and the subject is administered Olaparib at a dose of about 8 mg/kg/day. In some embodiments, the subject has melanoma, and the subject is administered Olaparib at a dose of about 9 mg/kg/day. In some embodiments, the subject has melanoma, and the subject is administered Olaparib at a dose of about 10 mg/kg/day. In some embodiments, the subject has melanoma, and the subject is administered Olaparib at a dose of about 15 mg/kg/day. In some embodiments, the subject has melanoma, and the subject is administered Olaparib at a dose of about 20 mg/kg/day. In some embodiments, the subject has melanoma, and the subject is administered Olaparib at a dose of about 25 mg/kg/day. In some embodiments, the subject has melanoma, and the subject is administered Olaparib at a dose of about 30 mg/kg/day. In some embodiments, the subject has melanoma, and the subject is administered Olaparib at a dose of about 40 mg/kg/day. In some embodiments, the subject has melanoma, and the subject is administered Olaparib at a dose of about 50 mg/kg/day. In some embodiments, Olaparib is administered per day orally.

In some embodiments, the subject has anal sac carcinoma, and the subject is administered Olaparib at a dose of about 0.01-0.1 mg/kg/day. In some embodiments, the subject has anal sac carcinoma, and the subject is administered Olaparib at a dose of about 1-20 mg/kg/day. In some embodiments, the subject has anal sac carcinoma, and the subject is administered Olaparib at a dose of about 5-10 mg/kg/day. In some embodiments, the subject has anal sac carcinoma, and the subject is administered Olaparib at a dose of about 1-5 mg/kg/day. In some embodiments, the subject has anal sac carcinoma, and the subject is administered Olaparib at a dose of about 0.01-0.05 mg/kg/day. In some embodiments, the subject has anal sac carcinoma, and the subject is administered Olaparib at a dose of about 0.02-0.03 mg/kg/day. In some embodiments, the subject has anal sac carcinoma, and the subject is administered Olaparib at a dose of about 0.1 mg/kg/day. In some embodiments, the subject has anal sac carcinoma, and the subject is administered Olaparib at a dose of about 0.02 mg/kg/day. In some embodiments, the subject has anal sac carcinoma, and the subject is administered Olaparib at a dose of about 0.03 mg/kg/day. In some embodiments, the subject has anal sac carcinoma, and the subject is administered Olaparib at a dose of about 1 mg/kg/day. In some embodiments, the subject has anal sac carcinoma, and the subject is administered Olaparib at a dose of about 2 mg/kg/day. In some embodiments, the subject has anal sac carcinoma, and the subject is administered Olaparib at a dose of about 3 mg/kg/day. In some embodiments, the subject has anal sac carcinoma, and the subject is administered Olaparib at a dose of about 4 mg/kg/day. In some embodiments, the subject has anal sac carcinoma, and the subject is administered Olaparib at a dose of about 5 mg/kg/day. In some embodiments, the subject has anal sac carcinoma, and the subject is administered Olaparib at a dose of about 6 mg/kg/day. In some embodiments, the subject has anal sac carcinoma, and the subject is administered Olaparib at a dose of about 7 mg/kg/day. In some embodiments, the subject has anal sac carcinoma, and the subject is administered Olaparib at a dose of about 8 mg/kg/day. In some embodiments, the subject has anal sac carcinoma, and the subject is administered Olaparib at a dose of about 9 mg/kg/day. In some embodiments, the subject has anal sac carcinoma, and the subject is administered Olaparib at a dose of about 10 mg/kg/day. In some embodiments, the subject has anal sac carcinoma, and the subject is administered Olaparib at a dose of about 15 mg/kg/day. In some embodiments, the subject has anal sac carcinoma, and the subject is administered Olaparib at a dose of about 20 mg/kg/day. In some embodiments, the subject has anal sac carcinoma, and the subject is administered Olaparib at a dose of about 25 mg/kg/day. In some embodiments, the subject has anal sac carcinoma, and the subject is administered Olaparib at a dose of about 30 mg/kg/day. In some embodiments, the subject has anal sac carcinoma, and the subject is administered Olaparib at a dose of about 40 mg/kg/day. In some embodiments, the subject has anal sac carcinoma, and the subject is administered Olaparib at a dose of about 50 mg/kg/day. In some embodiments, Olaparib is administered per day orally.

In some embodiments, Toceranib is administered at a dose of about 0.01-0.1 mg/kg. In some embodiments, Toceranib is administered at a dose of about 1-20 mg/kg. In some embodiments, Toceranib is administered at a dose of about 5-10 mg/kg. In some embodiments, Toceranib is administered at a dose of about 2-3 mg/kg (e.g., about 2.5 mg/kg to about 2.75 mg/kg). In some embodiments, Toceranib is administered at a dose of about 0.01-0.05 mg/kg. In some embodiments, Toceranib is administered at a dose of about 0.02-0.03 mg/kg. In some embodiments, Toceranib is administered at a dose of about 0.1 mg/kg. In some embodiments, Toceranib is administered at a dose of about 0.02 mg/kg. In some embodiments, Toceranib is administered at a dose of about 0.03 mg/kg. In some embodiments, Toceranib is administered at a dose of about 1 mg/kg. In some embodiments, Toceranib is administered at a dose of about 2 mg/kg. In some embodiments, Toceranib is administered at a dose of about 3 mg/kg. In some embodiments, Toceranib is administered at a dose of about 4 mg/kg. In some embodiments, Toceranib is administered at a dose of about 5 mg/kg. In some embodiments, Toceranib is administered at a dose of about 6 mg/kg. In some embodiments, Toceranib is administered at a dose of about 7 mg/kg. In some embodiments, Toceranib is administered at a dose of about 8 mg/kg. In some embodiments, Toceranib is administered at a dose of about 9 mg/kg. In some embodiments, Toceranib is administered at a dose of about 10 mg/kg. In some embodiments, Toceranib is administered at a dose of about 15 mg/kg. In some embodiments, Toceranib is administered at a dose of about 20 mg/kg. In some embodiments, Toceranib is administered at a dose of about 25 mg/kg. In some embodiments, Toceranib is administered at a dose of about 30 mg/kg. In some embodiments, Toceranib is administered at a dose of about 40 mg/kg. In some embodiments, Toceranib is administered at a dose of about 50 mg/kg. In some embodiments, Toceranib is administered every other day. In some embodiments, Toceranib is administered three times a week (e.g., every Monday, Wednesday, and Friday).

In some embodiments, the subject has squamous cell carcinoma, and the subject is administered Toceranib at a dose of about 0.01-0.1 mg/kg. In some embodiments, the subject has squamous cell carcinoma, and the subject is administered Toceranib at a dose of about 1-20 mg/kg. In some embodiments, the subject has squamous cell carcinoma, and the subject is administered Toceranib at a dose of about 5-10 mg/kg. In some embodiments, the subject has squamous cell carcinoma, and the subject is administered Toceranib at a dose of about 2-3 mg/kg (e.g., about 2.5 mg/kg to about 2.75 mg/kg). In some embodiments, the subject has squamous cell carcinoma, and the subject is administered Toceranib at a dose of about 0.01-0.05 mg/kg. In some embodiments, the subject has squamous cell carcinoma, and the subject is administered Toceranib at a dose of about 0.02-0.03 mg/kg. In some embodiments, the subject has squamous cell carcinoma, and the subject is administered Toceranib at a dose of about 0.1 mg/kg. In some embodiments, the subject has squamous cell carcinoma, and the subject is administered Toceranib at a dose of about 0.02 mg/kg. In some embodiments, the subject has squamous cell carcinoma, and the subject is administered Toceranib at a dose of about 0.03 mg/kg. In some embodiments, the subject has squamous cell carcinoma, and the subject is administered Toceranib at a dose of about 1 mg/kg. In some embodiments, the subject has squamous cell carcinoma, and the subject is administered Toceranib at a dose of about 2 mg/kg. In some embodiments, the subject has squamous cell carcinoma, and the subject is administered Toceranib at a dose of about 3 mg/kg. In some embodiments, the subject has squamous cell carcinoma, and the subject is administered Toceranib at a dose of about 4 mg/kg. In some embodiments, the subject has squamous cell carcinoma, and the subject is administered Toceranib at a dose of about 5 mg/kg. In some embodiments, the subject has squamous cell carcinoma, and the subject is administered Toceranib at a dose of about 6 mg/kg. In some embodiments, the subject has squamous cell carcinoma, and the subject is administered Toceranib at a dose of about 7 mg/kg. In some embodiments, the subject has squamous cell carcinoma, and the subject is administered Toceranib at a dose of about 8 mg/kg. In some embodiments, the subject has squamous cell carcinoma, and the subject is administered Toceranib at a dose of about 9 mg/kg. In some embodiments, the subject has squamous cell carcinoma, and the subject is administered Toceranib at a dose of about 10 mg/kg. In some embodiments, the subject has squamous cell carcinoma, and the subject is administered Toceranib at a dose of about 15 mg/kg. In some embodiments, the subject has squamous cell carcinoma, and the subject is administered Toceranib at a dose of about 20 mg/kg. In some embodiments, the subject has squamous cell carcinoma, and the subject is administered Toceranib at a dose of about 25 mg/kg. In some embodiments, the subject has squamous cell carcinoma, and the subject is administered Toceranib at a dose of about 30 mg/kg. In some embodiments, the subject has squamous cell carcinoma, and the subject is administered Toceranib at a dose of about 40 mg/kg. In some embodiments, the subject has squamous cell carcinoma, and the subject is administered Toceranib at a dose of about 50 mg/kg. In some embodiments, Toceranib is administered every other day. In some embodiments, Toceranib is administered three times a week (e.g., every Monday, Wednesday, and Friday).

In some embodiments, Toceranib is administered at a dose of about 0.01-0.1 mg/kg/day. In some embodiments, Toceranib is administered at a dose of about 1-20 mg/kg/day. In some embodiments, Toceranib is administered at a dose of about 5-10 mg/kg/day. In some embodiments, Toceranib is administered at a dose of about 2-3 mg/kg/day (e.g., about 2.5 mg/kg/day to about 2.75 mg/kg/day). In some embodiments, Toceranib is administered at a dose of about 0.01-0.05 mg/kg/day. In some embodiments, Toceranib is administered at a dose of about 0.02-0.03 mg/kg/day. In some embodiments, Toceranib is administered at a dose of about 0.1 mg/kg/day. In some embodiments, Toceranib is administered at a dose of about 0.02 mg/kg/day. In some embodiments, Toceranib is administered at a dose of about 0.03 mg/kg/day. In some embodiments, Toceranib is administered at a dose of about 1 mg/kg/day. In some embodiments, Toceranib is administered at a dose of about 2 mg/kg/day. In some embodiments, Toceranib is administered at a dose of about 3 mg/kg/day. In some embodiments, Toceranib is administered at a dose of about 4 mg/kg/day. In some embodiments, Toceranib is administered at a dose of about 5 mg/kg/day. In some embodiments, Toceranib is administered at a dose of about 6 mg/kg/day. In some embodiments, Toceranib is administered at a dose of about 7 mg/kg/day. In some embodiments, Toceranib is administered at a dose of about 8 mg/kg/day. In some embodiments, Toceranib is administered at a dose of about 9 mg/kg/day. In some embodiments, Toceranib is administered at a dose of about 10 mg/kg/day. In some embodiments, Toceranib is administered at a dose of about 15 mg/kg/day. In some embodiments, Toceranib is administered at a dose of about 20 mg/kg/day. In some embodiments, Toceranib is administered at a dose of about 25 mg/kg/day. In some embodiments, Toceranib is administered at a dose of about 30 mg/kg/day. In some embodiments, Toceranib is administered at a dose of about 40 mg/kg/day. In some embodiments, Toceranib is administered at a dose of about 50 mg/kg/day. In some embodiments, Toceranib is administered every other day. In some embodiments, Toceranib is administered three times a week (e.g., every Monday, Wednesday, and Friday).

In some embodiments, the subject has squamous cell carcinoma, and the subject is administered Toceranib at a dose of about 0.01-0.1 mg/kg/day. In some embodiments, the subject has squamous cell carcinoma, and the subject is administered Toceranib at a dose of about 1-20 mg/kg/day. In some embodiments, the subject has squamous cell carcinoma, and the subject is administered Toceranib at a dose of about 5-10 mg/kg/day. In some embodiments, the subject has squamous cell carcinoma, and the subject is administered Toceranib at a dose of about 2-3 mg/kg/day (e.g., about 2.5 mg/kg/day to about 2.75 mg/kg/day). In some embodiments, the subject has squamous cell carcinoma, and the subject is administered Toceranib at a dose of about 0.01-0.05 mg/kg/day. In some embodiments, the subject has squamous cell carcinoma, and the subject is administered Toceranib at a dose of about 0.02-0.03 mg/kg/day. In some embodiments, the subject has squamous cell carcinoma, and the subject is administered Toceranib at a dose of about 0.1 mg/kg/day. In some embodiments, the subject has squamous cell carcinoma, and the subject is administered Toceranib at a dose of about 0.02 mg/kg/day. In some embodiments, the subject has squamous cell carcinoma, and the subject is administered Toceranib at a dose of about 0.03 mg/kg/day. In some embodiments, the subject has squamous cell carcinoma, and the subject is administered Toceranib at a dose of about 1 mg/kg/day. In some embodiments, the subject has squamous cell carcinoma, and the subject is administered Toceranib at a dose of about 2 mg/kg/day. In some embodiments, the subject has squamous cell carcinoma, and the subject is administered Toceranib at a dose of about 3 mg/kg/day. In some embodiments, the subject has squamous cell carcinoma, and the subject is administered Toceranib at a dose of about 4 mg/kg/day. In some embodiments, the subject has squamous cell carcinoma, and the subject is administered Toceranib at a dose of about 5 mg/kg/day. In some embodiments, the subject has squamous cell carcinoma, and the subject is administered Toceranib at a dose of about 6 mg/kg/day. In some embodiments, the subject has squamous cell carcinoma, and the subject is administered Toceranib at a dose of about 7 mg/kg/day. In some embodiments, the subject has squamous cell carcinoma, and the subject is administered Toceranib at a dose of about 8 mg/kg/day. In some embodiments, the subject has squamous cell carcinoma, and the subject is administered Toceranib at a dose of about 9 mg/kg/day. In some embodiments, the subject has squamous cell carcinoma, and the subject is administered Toceranib at a dose of about 10 mg/kg/day. In some embodiments, the subject has squamous cell carcinoma, and the subject is administered Toceranib at a dose of about 15 mg/kg/day. In some embodiments, the subject has squamous cell carcinoma, and the subject is administered Toceranib at a dose of about 20 mg/kg/day. In some embodiments, the subject has squamous cell carcinoma, and the subject is administered Toceranib at a dose of about 25 mg/kg/day. In some embodiments, the subject has squamous cell carcinoma, and the subject is administered Toceranib at a dose of about 30 mg/kg/day. In some embodiments, the subject has squamous cell carcinoma, and the subject is administered Toceranib at a dose of about 40 mg/kg/day. In some embodiments, the subject has squamous cell carcinoma, and the subject is administered Toceranib at a dose of about 50 mg/kg/day. In some embodiments, Toceranib is administered every other day. In some embodiments, Toceranib is administered three times a week (e.g., every Monday, Wednesday, and Friday).

In some embodiments, the subject has carcinoma, and the subject is administered Toceranib at a dose of about 0.01-0.1 mg/kg/day. In some embodiments, the subject has carcinoma, and the subject is administered Toceranib at a dose of about 1-20 mg/kg/day. In some embodiments, the subject has carcinoma, and the subject is administered Toceranib at a dose of about 5-10 mg/kg/day. In some embodiments, the subject has carcinoma, and the subject is administered Toceranib at a dose of about 2-3 mg/kg/day (e.g., about 2.5 mg/kg/day to about 2.75 mg/kg/day). In some embodiments, the subject has carcinoma, and the subject is administered Toceranib at a dose of about 0.01-0.05 mg/kg/day. In some embodiments, the subject has carcinoma, and the subject is administered Toceranib at a dose of about 0.02-0.03 mg/kg/day. In some embodiments, the subject has carcinoma, and the subject is administered Toceranib at a dose of about 0.1 mg/kg/day. In some embodiments, the subject has carcinoma, and the subject is administered Toceranib at a dose of about 0.02 mg/kg/day. In some embodiments, the subject has carcinoma, and the subject is administered Toceranib at a dose of about 0.03 mg/kg/day. In some embodiments, the subject has carcinoma, and the subject is administered Toceranib at a dose of about 1 mg/kg/day. In some embodiments, the subject has carcinoma, and the subject is administered Toceranib at a dose of about 2 mg/kg/day. In some embodiments, the subject has carcinoma, and the subject is administered Toceranib at a dose of about 3 mg/kg/day. In some embodiments, the subject has carcinoma, and the subject is administered Toceranib at a dose of about 4 mg/kg/day. In some embodiments, the subject has carcinoma, and the subject is administered Toceranib at a dose of about 5 mg/kg/day. In some embodiments, the subject has carcinoma, and the subject is administered Toceranib at a dose of about 6 mg/kg/day. In some embodiments, the subject has carcinoma, and the subject is administered Toceranib at a dose of about 7 mg/kg/day. In some embodiments, the subject has carcinoma, and the subject is administered Toceranib at a dose of about 8 mg/kg/day. In some embodiments, the subject has carcinoma, and the subject is administered Toceranib at a dose of about 9 mg/kg/day. In some embodiments, the subject has carcinoma, and the subject is administered Toceranib at a dose of about 10 mg/kg/day. In some embodiments, the subject has carcinoma, and the subject is administered Toceranib at a dose of about 15 mg/kg/day. In some embodiments, the subject has carcinoma, and the subject is administered Toceranib at a dose of about 20 mg/kg/day. In some embodiments, the subject has carcinoma, and the subject is administered Toceranib at a dose of about 25 mg/kg/day. In some embodiments, the subject has carcinoma, and the subject is administered Toceranib at a dose of about 30 mg/kg/day. In some embodiments, the subject has carcinoma, and the subject is administered Toceranib at a dose of about 40 mg/kg/day. In some embodiments, the subject has carcinoma, and the subject is administered Toceranib at a dose of about 50 mg/kg/day. In some embodiments, Toceranib is administered every other day. In some embodiments, Toceranib is administered three times a week (e.g., every Monday, Wednesday, and Friday).

In some embodiments, Palbociclib is administered at a dose of about 0.01-0.1 mg/kg. In some embodiments, Palbociclib is administered at a dose of about 0.05-0.4 mg/kg. In some embodiments, Palbociclib is administered at a dose of about 1-20 mg/kg. In some embodiments, Palbociclib is administered at a dose of about 5-10 mg/kg. In some embodiments, Palbociclib is administered at a dose of about 0.01-0.05 mg/kg. In some embodiments, Palbociclib is administered at a dose of about 0.02-0.03 mg/kg. In some embodiments, Palbociclib is administered at a dose of about 0.1 mg/kg. In some embodiments, Palbociclib is administered at a dose of about 0.2 mg/kg. In some embodiments, Palbociclib is administered at a dose of about 0.02 mg/kg. In some embodiments, Palbociclib is administered at a dose of about 0.03 mg/kg. In some embodiments, Palbociclib is administered at a dose of about 5 mg/kg. In some embodiments, Palbociclib is administered at a dose of about 6 mg/kg. In some embodiments, Palbociclib is administered at a dose of about 7 mg/kg. In some embodiments, Palbociclib is administered at a dose of about 8 mg/kg. In some embodiments, Palbociclib is administered at a dose of about 9 mg/kg. In some embodiments, Palbociclib is administered at a dose of about 10 mg/kg. In some embodiments, Palbociclib is administered at a dose of about 15 mg/kg. In some embodiments, Palbociclib is administered at a dose of about 20 mg/kg. In some embodiments, Palbociclib is administered at a dose of about 25 mg/kg. In some embodiments, Palbociclib is administered at a dose of about 30 mg/kg. In some embodiments, Palbociclib is administered at a dose of about 40 mg/kg. In some embodiments, Palbociclib is administered at a dose of about 50 mg/kg. In some embodiments, Palbociclib is administered per day orally.

In some embodiments, the subject has melanoma, and the subject is administered Palbociclib at a dose of about 0.01-0.1 mg/kg. In some embodiments, the subject has melanoma, and the subject is administered Palbociclib at a dose of about 0.05-0.4 mg/kg. In some embodiments, the subject has melanoma, and the subject is administered Palbociclib at a dose of about 1-20 mg/kg. In some embodiments, the subject has melanoma, and the subject is administered Palbociclib at a dose of about 5-10 mg/kg. In some embodiments, the subject has melanoma, and the subject is administered Palbociclib at a dose of about 0.01-0.05 mg/kg. In some embodiments, the subject has melanoma, and the subject is administered Palbociclib at a dose of about 0.02-0.03 mg/kg. In some embodiments, the subject has melanoma, and the subject is administered Palbociclib at a dose of about 0.1 mg/kg. In some embodiments, the subject has melanoma, and the subject is administered Palbociclib at a dose of about 0.2 mg/kg. In some embodiments, the subject has melanoma, and the subject is administered Palbociclib at a dose of about 0.02 mg/kg. In some embodiments, the subject has melanoma, and the subject is administered Palbociclib at a dose of about 0.03 mg/kg. In some embodiments, the subject has melanoma, and the subject is administered Palbociclib at a dose of about 5 mg/kg. In some embodiments, the subject has melanoma, and the subject is administered Palbociclib at a dose of about 6 mg/kg. In some embodiments, the subject has melanoma, and the subject is administered Palbociclib at a dose of about 7 mg/kg. In some embodiments, the subject has melanoma, and the subject is administered Palbociclib at a dose of about 8 mg/kg. In some embodiments, the subject has melanoma, and the subject is administered Palbociclib at a dose of about 9 mg/kg. In some embodiments, the subject has melanoma, and the subject is administered Palbociclib at a dose of about 10 mg/kg. In some embodiments, the subject has melanoma, and the subject is administered Palbociclib at a dose of about 15 mg/kg. In some embodiments, the subject has melanoma, and the subject is administered Palbociclib at a dose of about 20 mg/kg. In some embodiments, the subject has melanoma, and the subject is administered Palbociclib at a dose of about 25 mg/kg. In some embodiments, the subject has melanoma, and the subject is administered Palbociclib at a dose of about 30 mg/kg. In some embodiments, the subject has melanoma, and the subject is administered Palbociclib at a dose of about 40 mg/kg. In some embodiments, the subject has melanoma, and the subject is administered Palbociclib at a dose of about 50 mg/kg. In some embodiments, Palbociclib is administered per day orally.

In some embodiments, Palbociclib is administered at a dose of about 0.01-0.1 mg/kg/day. In some embodiments, Palbociclib is administered at a dose of about 0.05-0.4 mg/kg/day. In some embodiments, Palbociclib is administered at a dose of about 1-20 mg/kg/day. In some embodiments, Palbociclib is administered at a dose of about 5-10 mg/kg/day. In some embodiments, Palbociclib is administered at a dose of about 0.01-0.05 mg/kg/day. In some embodiments, Palbociclib is administered at a dose of about 0.02-0.03 mg/kg/day. In some embodiments, Palbociclib is administered at a dose of about 0.1 mg/kg/day. In some embodiments, Palbociclib is administered at a dose of about 0.2 mg/kg/day. In some embodiments, Palbociclib is administered at a dose of about 0.02 mg/kg/day. In some embodiments, Palbociclib is administered at a dose of about 0.03 mg/kg/day. In some embodiments, Palbociclib is administered at a dose of about 5 mg/kg/day. In some embodiments, Palbociclib is administered at a dose of about 6 mg/kg/day. In some embodiments, Palbociclib is administered at a dose of about 7 mg/kg/day. In some embodiments, Palbociclib is administered at a dose of about 8 mg/kg/day. In some embodiments, Palbociclib is administered at a dose of about 9 mg/kg/day. In some embodiments, Palbociclib is administered at a dose of about 10 mg/kg/day. In some embodiments, Palbociclib is administered at a dose of about 15 mg/kg/day. In some embodiments, Palbociclib is administered at a dose of about 20 mg/kg/day. In some embodiments, Palbociclib is administered at a dose of about 25 mg/kg/day. In some embodiments, Palbociclib is administered at a dose of about 30 mg/kg/day. In some embodiments, Palbociclib is administered at a dose of about 40 mg/kg/day. In some embodiments, Palbociclib is administered at a dose of about 50 mg/kg/day. In some embodiments, Palbociclib is administered per day orally.

In some embodiments, the subject has melanoma (e.g., malignant melanoma), and the subject is administered Palbociclib at a dose of about 0.01-0.1 mg/kg/day. In some embodiments, the subject has melanoma, and the subject is administered Palbociclib at a dose of about 0.05-0.4 mg/kg/day. In some embodiments, the subject has melanoma, and the subject is administered Palbociclib at a dose of about 1-20 mg/kg/day. In some embodiments, the subject has melanoma, and the subject is administered Palbociclib at a dose of about 5-10 mg/kg/day. In some embodiments, the subject has melanoma, and the subject is administered Palbociclib at a dose of about 0.01-0.05 mg/kg/day. In some embodiments, the subject has melanoma, and the subject is administered Palbociclib at a dose of about 0.02-0.03 mg/kg/day. In some embodiments, the subject has melanoma, and the subject is administered Palbociclib at a dose of about 0.1 mg/kg/day. In some embodiments, the subject has melanoma, and the subject is administered Palbociclib at a dose of about 0.2 mg/kg/day. In some embodiments, the subject has melanoma, and the subject is administered Palbociclib at a dose of about 0.02 mg/kg/day. In some embodiments, the subject has melanoma, and the subject is administered Palbociclib at a dose of about 0.03 mg/kg/day. In some embodiments, the subject has melanoma, and the subject is administered Palbociclib at a dose of about 5 mg/kg/day. In some embodiments, the subject has melanoma, and the subject is administered Palbociclib at a dose of about 6 mg/kg/day. In some embodiments, the subject has melanoma, and the subject is administered Palbociclib at a dose of about 7 mg/kg/day. In some embodiments, the subject has melanoma, and the subject is administered Palbociclib at a dose of about 8 mg/kg/day. In some embodiments, the subject has melanoma, and the subject is administered Palbociclib at a dose of about 9 mg/kg/day. In some embodiments, the subject has melanoma, and the subject is administered Palbociclib at a dose of about 10 mg/kg/day. In some embodiments, the subject has melanoma, and the subject is administered Palbociclib at a dose of about 15 mg/kg/day. In some embodiments, the subject has melanoma, and the subject is administered Palbociclib at a dose of about 20 mg/kg/day. In some embodiments, the subject has melanoma, and the subject is administered Palbociclib at a dose of about 25 mg/kg/day. In some embodiments, the subject has melanoma, and the subject is administered Palbociclib at a dose of about 30 mg/kg/day. In some embodiments, the subject has melanoma, and the subject is administered Palbociclib at a dose of about 40 mg/kg/day. In some embodiments, the subject has melanoma, and the subject is administered Palbociclib at a dose of about 50 mg/kg/day. In some embodiments, Palbociclib is administered per day orally.

### Diagnostic Assays

In some embodiments, described herein are methods of identifying subjects with cancer that will respond to pharmaceutical compositions comprising a compound described herein. In various embodiments, described herein are methods of treating cancer in a subject (e.g., a canine subject) comprising administration of pharmaceutical compositions comprising a compound described herein and further comprising the step of determining or having determined if the cancer harbors at least one mutation in one or more genes prior to the administration of the pharmaceutical composition comprising a compound described herein. In various embodiments, described herein are methods of treating cancer in a canine subject comprising administration of pharmaceutical compositions comprising a compound described herein and further comprising the step of determining or having determined if the cancer overexpresses or under expresses one or more genes compared to non-cancerous tissue prior to the administration of the pharmaceutical composition comprising a compound described herein.

In some embodiments, the determining at least one mutation in at least one gene is determined from a biological sample derived from the cancer. In certain embodiments, the biological sample comprises nucleic acid. The nucleic acid can be DNA, RNA or combinations thereof. Any method known in the art can be used to determine the sequence of a gene. In certain embodiments, the determining is performed by sequencing nucleic acid.

In some embodiments, the mutation is a splice variant, a frameshift, a missense, or a nonsense mutation.

In some embodiments, the at least one mutation is a mutation in p53 (TP53). In certain aspects, the at least one mutation is a mutation in BRCA1. In some embodiments, the at least one mutation is a mutation in the retinoblastoma gene (RB1).

In some embodiments, the biological sample is derived from a subject with osteosarcoma, it is determined that the cancer harbors a mutation in p53, and the subject is selected for treatment with Olaparib. In some embodiments, the biological sample is derived from a subject with osteosarcoma, it is determined that the cancer harbors a mutation in BRCA1 and/or BRCA2, and the subject is selected for treatment with Olaparib. In some embodiments, the biological sample is derived from a subject with osteosarcoma, it is determined that the cancer harbors a mutation in BRCA1, and the subject is selected for treatment with Olaparib. In some embodiments, the biological sample is derived from a subject with osteosarcoma, it is determined that the cancer harbors a mutation in BRCA2, and the subject is selected for treatment with Olaparib. In some embodiments, the biological sample is derived from a subject with osteosarcoma, it is determined that the cancer harbors a mutation in BRCA1 and BRCA2, and the subject is selected for treatment with Olaparib. In some embodiments, the biological sample is derived from a subject with osteosarcoma, it is determined that the cancer harbors a mutation in RB1, and the subject is selected for treatment with Olaparib.

In some embodiments, the biological sample is derived from a subject with soft tissue sarcoma, it is determined that the cancer harbors a mutation in p53, and the subject is selected for treatment with Olaparib.

In some embodiments, the biological sample is derived from a subject with melanoma (e.g., oral malignant melanoma), it is determined that the cancer harbors a mutation in BRCA1 and/or BRCA2, and the subject is selected for treatment with Olaparib. In some embodiments, the biological sample is derived from a subject with melanoma, it is determined that the cancer harbors a mutation in BRCA1, and the subject is selected for treatment with Olaparib. In some embodiments, the biological sample is derived from a subject with melanoma, it is determined that the cancer harbors a mutation in BRCA2, and the subject is selected for treatment with Olaparib. In some embodiments, the biological sample is derived from a subject with melanoma, it is determined that the cancer harbors a mutation in BRCA1 and BRCA2, and the subject is selected for treatment with Olaparib.

In some embodiments, the biological sample is derived from a subject with osteosarcoma, it is determined that the cancer harbors a mutation in p53, and the subject is selected for treatment with Rapamycin. In some embodiments, the biological sample is derived from a subject with osteosarcoma, it is determined that the cancer harbors a mutation in BRCA1, and the subject is selected for treatment with Rapamycin. In some embodiments, the biological sample is derived from a subject with osteosarcoma, it is determined that the cancer harbors a mutation in RB1, and the subject is selected for treatment with Rapamycin.

In some embodiments, the biological sample is derived from a subject with osteosarcoma, it is determined that the cancer harbors a mutation in p53, and the subject is selected for treatment with Olaparib and Rapamycin. In some embodiments, the biological sample is derived from a subject with osteosarcoma, it is determined that the cancer harbors a mutation in BRCA1, and the subject is selected for treatment with Olaparib and Rapamycin. In some embodiments, the biological sample is derived from a subject with osteosarcoma, it is determined that the cancer harbors a mutation in RB1, and the subject is selected for treatment with Olaparib and Rapamycin.

In some embodiments, the biological sample is derived from a subject with melanoma (e.g., malignant melanoma), it is determined that the cancer harbors a mutation in CDK4, and the subject is selected for treatment with Palbociclib.

### Combination Therapies

Administration of the pharmaceutical compositions described herein comprising a compound described herein can be concurrent with (at the same time), sequential to (at a different time but on the same day, e.g., during the same subject visit), or separate from (on a different day) administration of another anti-cancer agent or therapy. When administered sequentially or separately, the pharmaceutical compositions comprising a compound described herein can be administered before, after, or both before and after the other anti-cancer agent or therapy. In certain embodiments, the additional therapy is surgery. In certain embodiments, the additional therapy is administration of ionizing radiation to the subject.

### Anti-Cancer Agents

The pharmaceutical compositions described herein can be administered alone or in combination with other treatments, either simultaneously or sequentially dependent upon the condition to be treated. One or more additional anti-cancer agents may be administered to the subject. Anti-cancer agents can be, but are not limited to, chemotherapeutic agents, targeted therapies, hormones, and immunotherapies. Anti-cancer agents can be, but are not limited to, a small molecule, an antibody or antibody fragment, nucleic acid (e.g., DNA or RNA), carbohydrates, peptides, lipids, exosomes, cells, or combinations thereof.

Chemotherapeutic agents include, but are not limited to alkylating agents, antimetabolites, anti-tumor antibiotics (e.g., doxorubicin, daunorubicin, bleomycin, dactinomycin), topoisomerase inhibitors (e.g., etoposide, irinotecan, topotecan), and mitotic inhibitors (e.g., docetaxel, eribulin, ixabepilone, paclitaxel, vinblastine).

Examples of anti-cancer agents include, but are not limited to: Abemaciclib, Abiraterone Acetate, Abraxane (Paclitaxel Albumin-stabilized Nanoparticle Formulation), Acalabrutinib, Actemra (Tocilizumab), Adcetris (Brentuximab Vedotin), Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alpelisib, Alunbrig (Brigatinib), Ameluz (Aminolevulinic Acid Hydrochloride), Amifostine, Aminolevulinic Acid Hydrochloride, Anastrozole, Apalutamide, Aprepitant, Aranesp (Darbepoetin Alfa), Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Asparlas (Calaspargase Pegol-mknl), Atezolizumab, Avastin (Bevacizumab), Avelumab, Axicabtagene Ciloleucel, Axitinib, Azacitidine, Azedra (Iobenguane I 131), Balversa (Erdafitinib), Bavencio (Avelumab), Beleodaq (Belinostat), Belinostat, Bendamustine Hydrochloride, Bendeka (Bendamustine Hydrochloride), Besponsa (Inotuzumab Ozogamicin), Bevacizumab, Bexarotene, Bicalutamide, BiCNU (Carmustine), Binimetinib, Bleomycin Sulfate, Blinatumomab, Blincyto (Blinatumomab), Bortezomib, Bosulif (Bosutinib), Bosutinib, Braftovi (Encorafenib), Brentuximab Vedotin, Brigatinib, BuMel, Busulfan, Busulfex (Busulfan), Cabazitaxel, Cablivi (Caplacizumab-yhdp), Cabometyx (Cabozantinib-S-Malate), Cabozantinib-S-Malate, Calaspargase Pegol-mknl, Calquence (Acalabrutinib), Campath (Alemtuzumab), Camptosar (Irinotecan Hydrochloride), Capecitabine, Caplacizumab-yhdp, Carac (Fluorouracil—Topical), Carboplatin, Carfilzomib, Carmustine, Carmustine Implant, Casodex (Bicalutamide), Cemiplimab-rwlc, Ceritinib, Cerubidine (Daunorubicin Hydrochloride), Cervarix (Recombinant HPV Bivalent Vaccine), Cetuximab, Chlorambucil, Cisplatin, Cladribine, Clofarabine, Clolar (Clofarabine), Cobimetinib, Cometriq (Cabozantinib-S-Malate), Copanlisib Hydrochloride, Copiktra (Duvelisib), Cosmegen (Dactinomycin), Cotellic (Cobimetinib), Crizotinib, Cyclophosphamide, Cyramza (Ramucirumab), Cytarabine, Cytarabine Liposome, Dabrafenib Mesylate, Dacarbazine, Dacogen (Decitabine), Dacomitinib, Dactinomycin, Daratumumab, Darbepoetin Alfa, Darzalex (Daratumumab), Dasatinib, Daunorubicin Hydrochloride, Daunorubicin Hydrochloride and Cytarabine Liposome, Daurismo (Glasdegib Maleate), Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Durvalumab, Duvelisib, Efudex (Fluorouracil—Topical), Eligard (Leuprolide Acetate), Elitek (Rasburicase), Ellence (Epirubicin Hydrochloride), Elotuzumab, Eloxatin (Oxaliplatin), Eltrombopag Olamine, Elzonris (Tagraxofusp-erzs), Emapalumab-lzsg, Emend (Aprepitant), Empliciti (Elotuzumab), Enasidenib Mesylate, Encorafenib, Enzalutamide, Epirubicin Hydrochloride, Epoetin Alfa, Epogen (Epoetin Alfa), Erbitux (Cetuximab), Erdafitinib, Eribulin Mesylate, Erivedge (Vismodegib), Erleada (Apalutamide), Erlotinib Hydrochloride, Erwinaze (Asparaginase Erwinia chrysanthemi), Ethyol (Amifostine) Etopophos (Etoposide Phosphate), Etoposide, Etoposide Phosphate, Everolimus Evista (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), Femara (Letrozole), Filgrastim, Firmagon (Degarelix), Fludarabine Phosphate, Fluoroplex (Fluorouracil—Topical), Fluorouracil Injection, Fluorouracil-Topical, Flutamide, Folotyn (Pralatrexate), Fostamatinib Disodium, Fulvestrant, Fusilev (Leucovorin Calcium), Gamifant (Emapalumab-lzsg), Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, Gemtuzumab Ozogamicin, Gemzar (Gemcitabine Hydrochloride), Gilotrif (Afatinib Dimaleate), Gilteritinib Fumarate, Glasdegib Maleate, Gleevec (Imatinib Mesylate), Gliadel Wafer (Carmustine Implant), Glucarpidase, Goserelin Acetate, Granisetron, Granisetron Hydrochloride, Granix (Filgrastim), Halaven (Eribulin Mesylate), Hemangeol (Propranolol Hydrochloride), Herceptin Hylecta (Trastuzumab and Hyaluronidase-oysk), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Ibrance (Palbociclib), Ibritumomab Tiuxetan, Ibrutinib, Iclusig (Ponatinib Hydrochloride), Idamycin PFS (Idarubicin Hydrochloride), Idarubicin Hydrochloride, Idelalisib, Idhifa (Enasidenib Mesylate), Ifex (Ifosfamide), Ifosfamide, IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iobenguane I 131, Ipilimumab, Iressa (Gefitinib), Irinotecan Hydrochloride, Irinotecan Hydrochloride Liposome, Istodax (Romidepsin), Ivosidenib, Ixabepilone, Ixazomib Citrate, Ixempra (Ixabepilone), Jakafi (Ruxolitinib Phosphate), Jevtana (Cabazitaxel), Kadcyla (Ado-Trastuzumab Emtansine), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Larotrectinib Sulfate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Levulan Kerastik (Aminolevulinic Acid Hydrochloride), Libtayo (Cemiplimab-rwlc), Lomustine, Lonsurf (Trifluridine and Tipiracil Hydrochloride), Lorbrena (Lorlatinib), Lorlatinib, Lumoxiti (Moxetumomab Pasudotox-tdfk), Lupron (Leuprolide Acetate), Lupron Depot (Leuprolide Acetate), Lutathera (Lutetium Lu 177—Dotatate), Lutetium (Lu 177—Dotatate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Mektovi (Binimetinib), Melphalan, Melphalan Hydrochloride, Mercaptopurine, Mesna, Mesnex (Mesna), Methotrexate, Methylnaltrexone Bromide, Midostaurin, Mitomycin C, Mitoxantrone Hydrochloride, Mogamulizumab-kpkc, Moxetumomab Pasudotox-tdfk, Mozobil (Plerixafor), Mustargen (Mechlorethamine Hydrochloride), Mvasi (Bevacizumab), Myleran (Busulfan), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nplate (Romiplostim), Obinutuzumab, Odomzo (Sonidegib), Ofatumumab, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), Osimertinib Mesylate, Oxaliplatin, Paclitaxel, Paclitaxel Albumin-stabilized Nanoparticle Formulation Palbociclib, Palifermin, Palonosetron Hydrochloride, Palonosetron Hydrochloride and Netupitant, Pamidronate Disodium, Panitumumab, Panobinostat, Pazopanib Hydrochloride, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Piqray (Alpelisib), Plerixafor, Polatuzumab Vedotin-piiq, Polivy (Polatuzumab Vedotin-piiq), Pomalidomide, Pomalyst (Pomalidomide), Ponatinib Hydrochloride, Portrazza (Necitumumab), Poteligeo (Mogamulizumab-kpkc), Pralatrexate, Prednisone, Procarbazine Hydrochloride, Procrit (Epoetin Alfa), Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, Ravulizumab-cwvz, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), Retacrit (Epoetin Alfa), Revlimid (Lenalidomide), Rheumatrex (Methotrexate), Ribociclib, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sancuso (Granisetron), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), Sterile Talc Powder (Talc), Steritalc (Talc), Stivarga (Regorafenib), Sunitinib Malate, Sustol (Granisetron), Sutent (Sunitinib Malate), Sylatron (Peginterferon Alfa-2b), Sylvant (Siltuximab), Synribo (Omacetaxine Mepesuccinate), Tabloid (Thioguanine), Tafinlar (Dabrafenib Mesylate), Tagraxofusp-erzs, Tagrisso (Osimertinib Mesylate), Talazoparib Tosylate, Talc, Talimogene Laherparepvec, Talzenna (Talazoparib Tosylate), Tamoxifen Citrate, Tarceva (Erlotinib Hydrochloride), Targretin (Bexarotene), Tasigna (Nilotinib), Tavalisse (Fostamatinib Disodium), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tibsovo (Ivosidenib), Tisagenlecleucel, Tocilizumab, Tolak (Fluorouracil—Topical), Topotecan Hydrochloride, Toremifene, Torisel (Temsirolimus), Totect (Dexrazoxane Hydrochloride), Trabectedin, Trametinib, Trastuzumab, Trastuzumab and Hyaluronidase-oysk, Treanda (Bendamustine Hydrochloride), Trexall (Methotrexate), Trifluridine and Tipiracil Hydrochloride, Trisenox (Arsenic Trioxide), Tykerb (Lapatinib Ditosylate), Ultomiris (Ravulizumab-cwvz), Unituxin (Dinutuximab), Uridine Triacetate, Valrubicin, Valstar (Valrubicin), Vandetanib, Varubi (Rolapitant Hydrochloride), Vectibix (Panitumumab), VeIP, Velcade (Bortezomib), Vemurafenib, Venclexta (Venetoclax), Venetoclax, Verzenio (Abemaciclib), Vidaza (Azacitidine), Vinblastine Sulfate, Vincristine Sulfate, Vincristine Sulfate Liposome, Vinorelbine Tartrate, Vismodegib, Vistogard (Uridine Triacetate), Vitrakvi (Larotrectinib Sulfate), Vizimpro (Dacomitinib), Voraxaze (Glucarpidase), Vorinostat, Votrient (Pazopanib Hydrochloride), Vyxeos (Daunorubicin Hydrochloride and Cytarabine Liposome), Xalkori (Crizotinib), Xeloda (Capecitabine), Xgeva (Denosumab), Xofigo (Radium 223 Dichloride), Xospata (Gilteritinib Fumarate), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), Zytiga (Abiraterone Acetate), and derivatives thereof. In some embodiments, the anti-cancer agent is selected from the group consisting of: Crizotinib, Erlotinib, Gefinitib, Imatinib, Dasatinib, Rapamycin, Sorafenib, Trametinib, and Vorinostat. In some embodiments, the anti-cancer agent is selected from the group consisting of: carboplatin, cisplatin, and oxaliplatin. In some embodiments, the anti-cancer agent is carboplatin.

### Pharmaceutical Compositions

Methods for treatment of cancer and hyperproliferative diseases described herein include administering a therapeutically effective amount of a compound described herein. A compound described herein can be formulated in pharmaceutical compositions. Additionally, in certain embodiments, the subject is administered one or more additional pharmaceutical compositions comprising one or more additional anti-cancer agents. These compositions can comprise, in addition to a compound described herein and/or anti-cancer agent, a pharmaceutically acceptable excipient, carrier, buffer, stabiliser or other materials well known to those skilled in the art. Such materials should be non-toxic and should not interfere with the efficacy of the active ingredient. The precise nature of the carrier or other material can depend on the route of administration, e.g., oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

The pharmaceutically useful compound according to the present disclosure that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc., is within the responsibility of general practitioners and other medical doctors, and typically takes account of the disorder to be treated, the condition of the individual subject, the site of delivery, the method of administration and other factors known to practitioners. Examples of the techniques and protocols mentioned above can be found in Remington's Pharmaceutical Sciences, 16th edition, Osol, A. (ed), 1980.

### EXAMPLES

Below are examples of specific embodiments for carrying out the methods described in this disclosure. The examples are offered for illustrative purposes only and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Treatment of Cancers with Olaparib, Toceranib, and Palbociclib

This Example demonstrates efficacy of Olaparib, Toceranib, and Palbociclib in treating osteosarcoma, squamous cell carcinoma, and melanoma, respectively.

A Cox proportional-hazards model was used to fit survival time. The model estimates the risk of death at a given time, h(t|X)=h(t)*exp(X₁β₁+ . . . +Xₚβₚ), where h represents the risk of death at time t, and X represents p covariates. Additionally, the ratio of the hazard rates in the treatment group versus the control group is represented as the hazard ratio.

A one-hot encoded feature for each targeted therapy (treatment) received by the dog was created. For example, if a dog receives multiple treatments, the encoding for each treatment would be a “1,” otherwise, a treatment is encoded as “0”. Similarly, gene mutations were one-hot encoded and treated as independent variables during the analyses. Tumor types were grouped into broad categories to preserve sample size and include them as one-hot encoded features.

Each dog's survival time and status were determined based on their last known update, which was reported by the veterinarian's office. A dog is only considered deceased if there is a reported date of death, and is only considered to have survived up until the last known update. Two survival intervals were computed: survival from time of diagnosis and survival from time of treatment. When reporting hazard ratios regardless of intervention, survival time is defined as survival from time of diagnosis, which is the time from diagnosis to last known update if the dog is still active, and the time from diagnosis to death if the dog is deceased. When reporting hazard ratios for specific treatments, survival time is defined as survival from treatment. The first date of any targeted therapy being ordered is used as the start of the treatment date.

Demographic features, including weight in kilograms, sex, and reproductive status (neutered or spayed vs intact), were extracted and used as covariates in the Cox model. In addition, to control for each dog's baseline survival rates, the dog's age at diagnosis, as well as the time from diagnosis to treatment are included, which is computed by subtracting the date that the targeted therapy was first ordered from the date of diagnosis. The data is seen in Table 1.

TABLE 1

| Data | Olaparib | Toceranib | Palbociclib |
|---|---|---|---|
| Tumor Types | Osteosarcoma | Squamous cell | Melanoma |
| | | carcinoma | |
| Hazard Ratio | 0.181 | 0.072 | 0.273 |
| (Signal Efficacy) | | | |
| p-Value | P = 0.021 | P = 0.005 | P = 0.043 |
| Median survival | Inf (16) | 577 d (13) | 433 d (13) |
| (#patients) with | | | |
| treatment | | | |
| Median survival | 154 d (158) | 183 d (32) | 159 d (114) |
| (#patients) without | | | |
| treatment | | | |

### Results

The median survival of subjects treated with Olaparib, Toceranib, and Palbociclib was improved with treatment as opposed to subjects not receiving treatment: no death observed vs. 154 days for Olaparib, 577 days vs. 183 days for Toceranib, and 433 days vs. 159 days for Palbociclib. The data is seen in Table 1.

The data shows that subject treated with Olaparib, Toceranib, and Palbociclib had improved survival than those that were not treated.

### Example 2: Biomarkers Associated with Prognosis and Treatment Prediction

This Example describes identification of biomarkers associated with prognosis and treatment prediction.

Briefly, real-world clinico-genomic data from 3000 dogs were analyzed. Specifically, Olaparib exhibited efficacy in TP53 and BRCA1-mutated cases (OS HR 0.34, P<0.001 and HR 0.39, P=0.004), while Rapamycin (mTOR inhibitor) demonstrated promising outcomes in TP53 and RB1-mutated canine tumors (OS HR 0.73, P=0.028 and HR 0.32, P=0.024). Further, TP53 mutant osteosarcomas exhibited improved prognosis with Olaparib treatment (OS HR 0.11, P<0.001), and soft tissue sarcomas with TP53 mutations demonstrated favorable responses to Rapamycin and Olaparib therapy (OS HR 0.11, P=0.012 and HR 0.07, P=0.011).

### Example 3: Treatment of Anal Sac Carcinoma and Melanoma with Olaparib

This Example demonstrates efficacy of Olaparib in treating anal sac carcinoma and melanoma.

A significant survival benefit in dogs with anal sac carcinoma treated with Olaparib was observed. Dogs receiving Olaparib achieved a median survival of 422 days compared to 273 days in untreated cases, with a hazard ratio of 0.319 and a statistically significant p-value of 0.023 (Table 2).

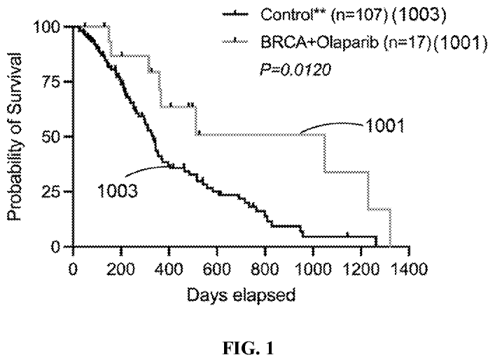

In dogs with oral malignant melanoma, treatment with Olaparib correlated with improved outcomes in cases exhibiting BRCA1 and BRCA2 mutations. Median survival reached 448 days in Olaparib-treated cases (gray, 1001) compared to 235 days for other biomarker profiles and alternative drugs (black, 1003) as seen in FIG. 1.

TABLE 2
Survival of dogs with anal sac carcinoma with Olaparib

| | Hazard | | Number of dogs | | Median Survival | |
|---|---|---|---|---|---|---|
| | Ratio | p- | With | Without | With | Without |
| Tumor | 95% CI | value | Olaparib | Olaparib | Olaparib | Olaparib |
| Anal Sac | 0.319 | 0.023 | 13 | 85 | 422 days | 372 days |
| carcinoma | (0.119- | | | | | |
| | 0.855) | | | | | |

### Example 4: Treatment of Carcinoma with Toceranib

This Example demonstrates efficacy of Toceranib in treating carcinoma.

Dogs receiving Toceranib achieved a median survival of 419 days compared to 250 days in untreated cases, with a hazard ratio of 0.292 and a statistically significant p-value of 0.003 (Table 3).

TABLE 3
Survival of dogs with carcinoma with Toceranib

| | Hazard | | Number of dogs | | Median Survival | |
|---|---|---|---|---|---|---|
| | Ratio | p- | With | Without | With | Without |
| Tumor | 95% CI | value | Toceranib | Toceranib | Toceranib | Toceranib |
| Carcinoma | 0.292 | 0.003 | 26 | 65 | 419 days | 250 days |
| | (1.31- | | | | | |
| | 0.65) | | | | | |

While the disclosure has been particularly shown and described with reference to a preferred embodiment and various alternate embodiments, it will be understood by persons skilled in the relevant art that various changes in form and details can be made therein without departing from the spirit and scope of the disclosure.

All references, issued patents and patent applications cited within the body of the instant specification are hereby incorporated by reference in their entirety, for all purposes.

1. A method of treating osteosarcoma in a canine subject, comprising: administering to the canine subject in need thereof a therapeutically effective amount of a pharmaceutical composition comprising Olaparib.

2. The method of claim 1, wherein the Olaparib is administered at a dose equal to or less than 5 mg/kg.

3. The method of claim 1, wherein the Olaparib is administered at a dose of 3 mg/kg.

4. The method of any one of claims 1-3, wherein the Olaparib is administered at a frequency selected from the group consisting of, twice daily, once daily, once every other day, once every third day, once every fourth day, once every 5^{th} day, or weekly.

5. The method of any one of claims 1-4, wherein the Olaparib is administered orally.

6. The method of any one of claims 1-5, further comprising administering a therapeutically effective amount of at least one additional anti-cancer agent.

7. The method of claim 6, wherein the at least one additional anti-cancer agent is a DNA damaging chemotherapeutic agent.

8. The method of claim 7, wherein the DNA damaging chemotherapeutic agent is selected from the group consisting of: a DNA-alkylating agent, DNA crosslinking agent, antimetabolite, topoisomerase inhibitor and a DNA intercalating agent.

9. The method of claim 6, wherein the at least one additional anti-cancer agent is a targeted anti-cancer agent.

10. The method of claim 9, wherein the targeted anti-cancer agent is selected from the group consisting of: Crizotinib, Erlotinib, Gefinitib, Imatinib, Dasatinib, Rapamycin, Sorafenib, Trametinib, and Vorinostat.

11. The method of claim 6, wherein the at least one additional anti-cancer agent is carboplatin.

12. The method of any one of claims 1-11, further comprising performing surgery on the subject.

13. The method of any one of claims 1-11, further comprising administering to the subject ionizing radiation.

14. The method of any one of claims 1-13, wherein the osteosarcoma harbors at least one mutation in at least one gene selected from the group consisting of p53 and BRCA1.

15. The method of any one of claims 1-13, further comprising having determined from a biological sample derived from the osteosarcoma, that the osteosarcoma harbors a mutation in at least one gene selected from the group consisting of p53 and BRCA1.

16. The method of claim 15, wherein the biological sample is a nucleic acid sample.

17. The method of claim 16, wherein the biological sample is a purified nucleic acid sample.

18. The method of claim 17, wherein the sample is DNA.

19. The method of claim 17, wherein the sample is RNA.

20. The method of claim 15, wherein the determining of the mutation in the at least one gene is performed by sequencing the nucleic acid sample.

21. A method of treating soft tissue sarcoma in a canine subject, comprising: administering to the canine subject in need thereof a therapeutically effective amount of a pharmaceutical composition comprising Olaparib.

22. The method of claim 21, wherein the Olaparib is administered at a dose equal to or less than 5 mg/kg.

23. The method of claim 21, wherein the Olaparib is administered at a dose of 3 mg/kg.

24. The method of any one of claims 21-23, wherein the Olaparib is administered at a frequency selected from the group consisting of, twice daily, once daily, once every other day, once every third day, once every fourth day, once every 5th day, or weekly.

25. The method of any one of claims 21-24, wherein the Olaparib is administered orally.

26. The method of any one of claims 21-25, further comprising administering a therapeutically effective amount of at least one additional anti-cancer agent.

27. The method of claim 26, wherein the at least one additional anti-cancer agent is a DNA damaging chemotherapeutic agent.

28. The method of claim 27, wherein the DNA damaging chemotherapeutic agent is selected from the group consisting of: a DNA-alkylating agent, DNA crosslinking agent, antimetabolite, topoisomerase inhibitor and a DNA intercalating agent.

29. The method of claim 26, wherein the at least one additional anti-cancer agent is a targeted anti-cancer agent.

30. The method of claim 29, wherein the targeted anti-cancer agent is selected from the group consisting of: Crizotinib, Erlotinib, Gefinitib, Imatinib, Dasatinib, Rapamycin, Sorafenib, Trametinib, and Vorinostat.

31. The method of claim 26, wherein the at least one additional anti-cancer agent is carboplatin.

32. The method of any one of claims 21-31, further comprising performing surgery on the subject.

33. The method of any one of claims 21-31, further comprising administering to the subject ionizing radiation.

34. The method of any one of claims 21-33, wherein the osteosarcoma harbors at least one mutation in at least one gene selected from the group consisting of p53 and BRCA1.

35. The method of any one of claims 21-33, further comprising having determined from a biological sample derived from the osteosarcoma, that the osteosarcoma harbors a mutation in at least one gene selected from the group consisting of p53 and BRCA1.

36. The method of claim 35, wherein the biological sample is a nucleic acid sample.

37. The method of claim 36, wherein the biological sample is a purified nucleic acid sample.

38. The method of claim 37, wherein the sample is DNA.

39. The method of claim 37, wherein the sample is RNA.

40. The method of claim 35, wherein the determining of the mutation in the at least one gene is performed by sequencing the nucleic acid sample.

41. A method of treating squamous cell carcinoma in a canine subject, comprising: administering to the canine subject in need thereof a therapeutically effective amount of a pharmaceutical composition comprising Toceranib.

42. The method of claim 41, wherein the Toceranib is administered at a dose equal to or less than 5 mg/kg.

43. The method of claim 41, wherein the Toceranib is administered at a dose 2 mg/kg to 3 mg/kg.

44. The method of any one of claims 42-43, wherein the Toceranib is administered at a frequency selected from the group consisting of, twice daily, once daily, once every other day, once every third day, once every fourth day, once every 5th day, or weekly.

45. The method of any one of claims 42-43, wherein the Toceranib is administered once three times a week.

46. The method of any one of claims 42-45, wherein the Toceranib is administered orally.

47. The method of any one of claims 42-46, further comprising administering a therapeutically effective amount of at least one additional anti-cancer agent.

48. The method of claim 47, wherein the at least one additional anti-cancer agent is a DNA damaging chemotherapeutic agent.

49. The method of claim 48, wherein the DNA damaging chemotherapeutic agent is selected from the group consisting of: a DNA-alkylating agent, DNA crosslinking agent, antimetabolite, topoisomerase inhibitor and a DNA intercalating agent.

50. The method of claim 47, wherein the at least one additional anti-cancer agent is a targeted anti-cancer agent.

51. The method of claim 50, wherein the targeted anti-cancer agent is selected from the group consisting of: Crizotinib, Erlotinib, Gefinitib, Imatinib, Dasatinib, Rapamycin, Sorafenib, Trametinib, and Vorinostat.

52. The method of any one of claims 41-51, further comprising performing surgery on the subject.

53. The method of any one of claims 41-51, further comprising administering to the subject ionizing radiation.

54. A method of treating melanoma in a canine subject, comprising: administering to the canine subject in need thereof a therapeutically effective amount of a pharmaceutical composition comprising Palbociclib.

55. The method of claim 54, wherein the Palbociclib is administered at a dose equal to or less than 0.2 mg/kg.

56. The method of claim 54, wherein the Palbociclib is administered at a dose of 0.05 mg/kg to 0.4 mg/kg.

57. The method of any one of claims 54-56, wherein the Palbociclib is administered at a frequency selected from the group consisting of, twice daily, once daily, once every other day, once every third day, once every fourth day, once every 5th day, or weekly.

58. The method of any one of claims 54-57, wherein the Palbociclib is administered orally.

59. The method of any one of claims 54-58, further comprising administering a therapeutically effective amount of at least one additional anti-cancer agent.

60. The method of claim 59, wherein the at least one additional anti-cancer agent is a DNA damaging chemotherapeutic agent.

61. The method of claim 60, wherein the DNA damaging chemotherapeutic agent is selected from the group consisting of: a DNA-alkylating agent, DNA crosslinking agent, antimetabolite, topoisomerase inhibitor and a DNA intercalating agent.

62. The method of claim 59, wherein the at least one additional anti-cancer agent is a targeted anti-cancer agent.

63. The method of claim 62, wherein the targeted anti-cancer agent is selected from the group consisting of: Crizotinib, Erlotinib, Gefinitib, Imatinib, Dasatinib, Rapamycin, Sorafenib, Trametinib, and Vorinostat.

64. The method of any one of claims 54-63, further comprising performing surgery on the subject.

65. The method of any one of claims 54-63, further comprising administering to the subject ionizing radiation.
